(19) United States
(12) Patent Application Publication
(10) Pub. No.: US 2012/0172415 A1
(43) Pub. Date: Jul. 5, 2012

(54) Exon Skipping Therapy for Functional Amelioration of Semifunctional Dystrophin in Becker and Duchenne Muscular Dystrophy

(76) Inventors: Thomas Voit, Paris (FR); Luis Garcia, Park (FR); Valerie Robin, Paris (FR); Patrick Dreyfus, Paris (FR)

(21) Appl. No.: 13/393,004

(22) PCT Filed: Aug. 31, 2010
(86) PCT No.: PCT/IB10/02339

§ 371 Date: Mar. 22, 2012

Related U.S. Application Data
(60) Provisional application No. 61/239,891, filed on Sep. 4, 2009.
(60) Provisional application No. 61/238,280, filed on Aug. 31, 2009.

(51) Int. Cl. A61K 31/7088; A61P 9/00; A61P 21/00; C12N 15/113; C07K 14/47

(52) U.S. Cl. 514/44 A; 536/24.5; 530/350

(57) ABSTRACT

Methods for stabilizing unstable proteins or for restoring functionality to non-functional or poorly functioning (semi-functional) proteins using exon skipping technology are provided. The methods involve the administration of antisense oligonucleotides to cause exon skipping, thereby removing one or more exons responsible for protein instability or lack of functionality. For example, exons encoding protease recognition sites may be removed. The method is useful for treating diseases caused by protein instability, such as Becker Muscular Dystrophy, or for treating Duchenne Muscular Distrophy patients with semi-functional dystrophin due to treatment with other exon skipping or stop codon readthrough therapies.

### BACKGROUND OF THE INVENTION

1. Field of the Invention

The invention generally relates to methods for stabilizing and/or restoring at least partial function to defective proteins using exon skipping technology. In particular, the invention provides methods to stabilize unstable dystrophin proteins by administering antisense oligonucleotides in order to cause exon skipping in order to treat Becker or Duchenne Muscular Dystrophy. More specifically, the invention relates to a method for stabilizing a semifunctional dystrophin that shows increased susceptibility for protein degradation by withdrawing, using exon skipping technology, a protease-sensitive site encoded by exon 42 of the dystrophin gene.

2. Background of the Invention

Duchenne Muscular Dystrophy (DMD) and Becker Muscular Dystrophy (BMD) are both genetic neuromuscular disorders due to mutations in the dystrophin gene. Dystrophin is a rod-shaped cytoplasmic protein, and a vital part of a protein complex that connects the cytoskeleton of a muscle fiber to the surrounding extracellular matrix through the cell membrane. Dystrophin is the longest gene known, covering 2.4 megabases (0.08% of the human genome) at locus Xp21. The primary transcript measures about 2,400 kilobases and the mature mRNA measures 14.0 kilobases. The 79 exons code for a protein of over 3500 amino acid residues.

DMD patients are characterized by a lack of dystrophin in muscles and present a severe phenotype, while BMD patients display a much milder phenotype and express either lower amounts of dystrophin or still semi-functional truncated dystrophins, hr some cases, such as in patients with deletion of exons 45-47 or 45-48 (about 50% of the BMD population), the resulting truncated dystrophins are unstable. These mutations are referred to herein as Δ45-47 and Δ45-48, respectively.

Currently, therapeutic strategies for DMD are being extensively explored, whereas the comprehension of the mechanisms responsible of BMD remains largely poorly investigated. There is a need to provide a better understanding of and viable treatment options for patients suffering from BMD.

In addition, novel therapeutic approaches currently in clinical trials use exon skipping strategies for the treatment of Duchenne muscular dystrophy. (van Deutekom, J et al, 2007; Kinali, M, et al, 2009) The restoration of the expression of a semifunctional dystrophin in these approaches is conferred by restoring the reading frame by introduction, using exon skipping, of an additional deletion of genetic material into the transcript. While such therapy offers some dystrophin functionality, unfortunately the semi-functional dystrophin molecules produced by such an approach also show increased susceptibility to protein degradation and are thus not fully functional. In addition, a semifunctional dystrophin that is the result of a stop codon readthrough therapy induced by medication such as PTC 124 (ataluren) is currently being tested in clinical trials (Welch, E M, et al, 2007). Again, while this treatment offers hope for some amelioration of disease symptoms, the semifunctional dystrophins that are produced exhibit the same undesirable susceptibility to protease degradation.

### SUMMARY OF THE INVENTION

In order to identify therapeutic avenues for DMD and BMD patients, the present invention takes into account a new understanding of the instability of semifunctional dystrophins. Semifunctional dystrophins include truncated dystrophins that are formed when some exons of the dystrophin gene are deleted, either due to an inherited mutation (for example, deletion of exons 45-47 or 45-48 as frequently found in BMD patients) or due to therapeutically induced deletions (e.g. deletion induced by exon skipping therapy that purposefully removes selected exons). Semifunctional dystrophins also include those that are produced as a result of stop codon readthrough therapy, as well as those produced as a result of various heritable mutations. The in viva instability of such semifunctional dystrophin proteins may be due to increased proteolytic susceptibility. Accordingly, the invention provides gene correction therapy and methods to increase the stability of semifunctional dystrophins by using exon skipping technology to remove exons which encode protease recognition sites, thereby enhancing stability of the dystrophin molecules. This is possible in part because the dystrophin protein contains several non-essential regions which can be removed without compromising the protein's function.

In particular, the invention provides methods to stabilize these truncated proteins by removing putative proteolytic cleavage sites using an antisense oligonucleotide (AON) mediated exon skipping strategy. In this approach, AONs are designed and used to cause “skipping” of one or more targeted exon(s) during pre-mRNA processing and thus to prevent inclusion of the amino acid sequences encoded by the exon(s) in the dystrophin protein, while retaining the open reading frame. This strategy allows the generation of internally deleted, but largely functional, dystrophin proteins. In one embodiment, a specific protease cleavage recognition sequence (site) encoded by nucleic acid sequences of exon 42 of the dystrophin gene was identified as theoretically “skippable” (the sequence HPSS in repeat 16). In order to test this hypothesis, exon 42 was eliminated from genes encoding Δ45-47 and Δ45-48 dystrophin proteins and the resulting Δ42, Δ45-47 and Δ42, Δ45-48 genes were translated in viva. The results showed that the Δ42, Δ45-47 and Δ42, Δ45-48 proteins, which lacked exon 42 and thus the protease cleavage site, were indeed stabilized in vivo relative to Δ45-47 and Δ45-48 dystrophin proteins, and displayed in vivo localization characteristics of normal dystrophin proteins.

The invention provides a method for treating a muscular dystrophy caused by instability of a semi-functional dystrophin in a patient in need thereof. The method comprises the step of administering to the patient antisense oligonucleotides complementary to nucleic acid sequences that are necessary for correct splicing of a region comprising or consisting of exon 42 of the semi-functional dystrophin in some embodiments, the antisense oligonucleotides are complementary to nucleic acid sequences within pre-mRNA encoding the one or more exons. In some embodiments, the antisense oligonucleotides are complementary to nucleic acid sequences adjacent to pre-mRNA encoding the one or more exons, said nucleic acid sequences adjacent to pre-mRNA encoding said one or more exons being required for correct splicing of the one or more exons. In one embodiment, the administered antisense oligonucleotides prevent cleavage of the semi-functional dystrophin at a protease recognition sequence HPSS. In one embodiment of the invention, the disease is Becker Muscular Dystrophy and the semi-functional dystrophin may be either Δ45-47 dystrophin or Δ45-48 dystrophin. In other embodiments, the disease is Duchenne Muscular Dystrophy with treatment-induced semifunctional dystrophin expression, and semifunctional dystrophin expression is induced by exon skipping treatment or by stop codon readthrough treatment. The antisense oligonucleotides may be administered to muscle tissue of said patient, for example, skeletal muscle tissue smooth muscle tissue and cardiac muscle tissue.

The invention also provides a method of stabilizing a semi-functional dystrophin protein. The method comprises the step of preventing cleavage of the semi-functional dystrophin protein at a protease recognition site HPSS (histidine, proline, serine, serine), in one embodiment, the step of preventing is carried out by blocking splicing of a region consisting of or comprising exon 42 of the semi-functional dystrophin. In some embodiments, the semi-functional dystrophin protein Δ45-47 dystrophin protein, Δ45-48 dystrophin protein, Δ49-51 dystrophin protein or Δ50-51 dystrophin protein.

The invention also provides antisense oligonucleotides complementary to nucleic acid sequences of exon 42 of dystrophin, or nucleic acid sequences adjacent to exon 42 which are required for correct splicing of exon 42 of dystrophin. In some embodiments, the antisense oligonucleotide is an oligomer which may be a phosphorodiamidate morpholino oligomer (PMO), a 2′-O-Met oligomer, a tricyclo (tc)-DNA oligomer, or a U1 or U7 short nuclear (sn) RNA oligomer.

The invention also provides a dystrophin protein which may be Δ42, Δ45-47 dystrophin protein; Δ42, Δ45-48 dystrophin protein; Δ42, Δ49-51 dystrophin protein; or Δ42, Δ50-51 dystrophin protein. The dystrophin protein may be an isolated and substantially purified protein.

In yet another embodiment, the invention provides a pharmaceutical composition containing one or more antisense oligonucleotides as described above for the treatment of a Becker or Duchenne Muscular Dystrophy. The antisense oligonucleotides are complementary to nucleic acid sequences of exon 42 of dystrophin, or nucleic acid sequences adjacent to exon 42 which are required for correct splicing of exon 42 of dystrophin. In some embodiments, the antisense oligonucleotides are oligomer which may be phosphorodiamidate morpholino oligomers (PMO), 2-O-Met oligomers, tricyclo (tc)-DNA oligomers, or U1 or U7 short nuclear (sn) RNA oligomers.

In another embodiment, the antisense oligonucleotides of the invention are for use in the manufacture of a medicament to treat a muscular dystrophy caused by instability of a semi-functional dystrophin; and/or for use in the treatment of a muscular dystrophy caused by instability of a semi-functional dystrophin. The muscular dystrophy that is treated may be, for example, a Becker or Duchenne Muscular Dystrophy. Methods of designing and manufacturing (making, synthesizing, etc.) antisense oligonucleotides to be used for (suitable for use in) the treatment of diseases in patients are known to those of skill in the art, as are methods for manufacturing compositions and formulations of oligonucleotides for administration. Such antisense oligonucleotides include the exemplary oligonucleotides presented herein, and others which will occur to those of skill in the art.

### BRIEF DESCRIPTION OF THE DRAWINGS

FIG. 1. Schematic representation of induction and differentiation of fibroblast-derived myogenic progenitor cells.

FIG. 2. PCR primers: black, antisense; single underline, SU7; outline, optimized sequence; double underline, restriction site.

FIG. 3A-C. Schematic of experimental protocol. A, cleavage of dystrophin cDNA and shuttle vector with restriction enzymes; B, PCR amplification; C, creation of GFP fusion protein with Δ45-47 deletion.

FIG. 4A-D. A and B: Multiplex Western blot of dystrophin from a, BMD patient with a Δ45-47 exon deletion; stained with A) DYS 1 antibody directed against the rod domain (exon 29) and B) DYS 2 antibody directed against the C-terminal domain of dystrophin. Both are multiplex blots also labeled for other antibodies such as calpain 3, dysferlin, α-sarcoglycan and γ-sarcoglycan. A dystrophin degradation band of 220 kD is visible with the DYS1 antibody. The total amount of full length dystrophin in the Δ45-47 BMD muscle is reduced due to protein degradation. C and D: Multiplex Western blot of dystrophin, calpain 3, α-sarcoglycan, γ-sarcoglycan and dysferlin, from C, BMD patients with a Δ45-48 exon deletion with antibody DYS1 (recognizes exon 29) compared to normal control; and D, BMD patients with a Δ45-48 exon deletion with antibody DYS 2 (recognizes the C-terminal domain) compared to normal control. The presence of a Δ45-48 exon deletion results in decreased full length dystrophin expression and appearance of an abnormal 220 KD dystrophin degradation band.

FIG. 5A-C. Localization of dystrophin and dystrophin deletion mutants in muscle fibers. A, localization of human full-length dystrophin-GFP at the tips of zebrafish muscle fibers; B, lack of accumulation of Δ45-47 human dystrophin-GFP at the tips of zebrafish muscle fibers; C, localization of Δ42, Δ45-47 dystrophin at the tips of zebrafish muscle fibers is similar to that of normal full-length dystrophin-GFP.

### DETAILED DESCRIPTION

As will be understood by those of skill in the art, in the cell nucleus, eukaryotic genes are transcribed into pre-messenger RNA (pre-mRNA) which contains both exons and introns. To form mature mRNA, splicing occurs at specific sequences at the borders of exons and introns (splice sites) thereby removing introns and connecting exons to one another to form mRNA, which is translated into protein. Exons can be specifically targeted to prevent their inclusion in mRNA using antisense oligonucleotides having sequences that are specifically complementary to sequences within or at the borders of a targeted exon e.g. complementary to splice donor or acceptor sites, which may include sequences internal to an exon or external and adjacent (usually 5′) to an exon. By annealing to these sequences, they interfere with the splicing machinery e.g. by overlapping and masking intron/exon splice junctions, thereby modifying splicing reactions so that the targeted exons are not included in the mature mRNA, i.e., the targeted exons are “skipped”. The mRNA thus no longer contains the information of the skipped exon and the protein it encodes does not contain an amino acid sequence corresponding to the skipped exon.

The invention provides methods for stabilizing proteins of interest that are otherwise prone to instability using exon skipping technology. The method involves blocking or preventing the incorporation into mature mRNA of one or more targeted exons which encode amino sequences that cause (are responsible for) the instability of the protein. This is accomplished by exposing the pre-mRNA that includes exons encoding the protein to antisense oligonucleotides (AONs) which are complementary to sequence motifs that are required for correct splicing of the one or more targeted exons. The AONs bind to complementary required sequences in the pre-mRNA and prevent normal splicing. Instead, the targeted exons are excised and are not included in the mature mRNA that is translated into protein, and the amino acid sequences encoded by the targeted exons are missing from the translated protein. In some embodiments of the invention, the instability of the proteins of interest is due to proteolytic degradation, and the one or more exons that are eliminated from the mRNA encode sequences that are protease recognition sequences. In one embodiment of the invention, the proteins of interest are Δ45-47 and Δ45-48 dystrophins and the exon that is removed is exon 42, which encodes the protein recognition sequence HPSS (SEQ ID NO: 1). The resulting Δ42, Δ45-47 and Δ42, Δ45-48 dystrophin proteins do not contain the unwanted proteolytic recognition sequence. As a result, the proteins are less susceptible to proteolytic degradation and are thus more stable within the cell. So long as the missing amino acids are otherwise not essential to the functioning of the dystrophin protein (e.g. if they are located in, for example, the central region of dystrophin) then the resulting shorter protein can still perform a stabilizing role in the muscle cell membrane.

In yet another embodiment the same invention introducing a Δ42 exon skip is used to stabilize a semifunctional dystrophin produced in DMD patients by the use of a therapeutic exon skipping strategy; or used to stabilize a semifunctional dystrophin that results from the readthrough of a stop codon in a DMD patient induced by medication such as PTC 124 or other substances revealing this property such as aminoglycosides (Barton-Davis, E R et al, 1999). In the latter therapeutic approach, the instability of the semifunctional dystrophin is the result of the haphazard inclusion of any one amino acid instead of the stop codon, thereby generating different dystrophin molecules in a cell of which at least some carry missense mutations, which may create or expose and make accessible a protease recognition site.

The invention provides methods of conferring stability on, or enhancing the stability of, or restoring partial or complete functionality to a protein, e.g. an unstable, defective, dysfunctional, partially functional, or nonfunctional protein. The protein is typically a mutant protein, the stability or function of which is attenuated or eliminated by one or more mutations. In particular, by “unstable” we mean the protein is susceptible to degradation by proteases, and particularly that the protein is more susceptible to protease degradation than its normal, non-mutant wild type form. The lack of stability and increased susceptibility to protease digestion may be due to any type of mutation (e.g. point mutation, deletion, insertion, etc.) that creates or exposes a protease recognition site that is not present or is not readily accessible in the non-mutant protein. Those of skill in the art will recognize that the stability of a protein in vivo may be linked to its function since a protein, to be fully functional and to carry out its intended role in a cell, must be present in a particular form, e.g. intact, properly folded, etc. Breaches of e.g. the peptide chain by protease cleavage can result in disassociation of a protein molecule, or partial unfolding, etc., thus attenuating or destroying function. In particular, experiments exploring the function of partially deleted micro- or mini-dystrophins have clearly shown that it is indispensable for the dystrophin molecule to be (semi-)functional to retain both the actin-binding N-terminal fraction as well as the plasma-membrane-binding C-terminus. (Li, S, et al, 2005; Gregorevic, P, et al, 2006). Disjunction by protease-mediated degradation therefore results invariably in loss of the dystrophin function. Those of skill in the art will recognize that the functioning of a protein can be damaged by mutations other than those that result from lack of stability due to protease susceptibility, e.g. by errors in protein folding, etc.

Those of skill in the art will recognize that there are many ways to determine or measure a level of stability or functionality of a protein, and to determine a level of increase or decrease of stability or functionality e.g. in response to a treatment protocol. Such methods include but are not limited to: measuring or detecting an amount of the intact protein or a related molecule (e.g. detecting the gene or a gene product); or measuring or detecting an activity of the protein, etc. Such measurements are generally made in comparison to a standard or control or “normal” sample. In addition, when the protein's lack of stability or functionality is involved in a disease process, disease symptoms may be monitored and/or measured (quantitated) in order to indirectly detect the presence or absence of a correctly functioning protein, or to gauge the success of a treatment protocol intended to remedy the lack of stability or functioning of the protein. In the present invention, a “semi-functional” protein is one which displays, for example, less than about 90%, or of less than about 80%, 70%, 60%, 50%, 40%, 30%, 20%, or even 10% or less of the normal function of the protein, when measured or quantitated by a method that is recognized in the art (e.g. ability, or lack thereof, to engage in actin-binding via an N-terminus and plasma membrane-binding via a C-terminus by one contiguous molecule; by measurements of localization and/or accumulation in muscle cells; by measuring or quantifying disease symptoms in patients, etc.). Also, a “semi-functional” dystrophin is any dystrophin which is produced in an individual with a BMD phenotype. In the present case, AONs are used to cause exon skipping resulting in an amelioration of disease symptoms (i.e. restoration of protein stability or protein function) in the range of at least about 10%, preferably about 20%, 30%, 40%, 50%, 60%, 70%, 80%, 90% or even 100%, compared to a non-treated control. Such symptoms may be observed on a micro level (e.g. N- and C-terminal binding, intracellular measurements as described above) or may be observed on a macro level (e.g. increase in strength, muscle mass, use of limbs, mobility; cardiac function, cognition, etc. in patients being treated).

In one embodiment of the invention, the protein that is stabilized or restored to function is the dystrophin protein, although this need not always be the case. Those of skill in the art will recognize that the techniques described herein are applicable to other eukaryotic proteins encoded by genes comprised of multiple exons and introns, and which are known to occur in mutant forms which are less stable than normal, wild type protein, due, for example, to the presence of an accessible proteolytic cleavage recognition site. The accessible recognition site may be present in non-mutant versions of the protein, or the recognition site may be created or exposed as a result of other mutations in the protein. Examples of other proteins for which the techniques of the invention may be used include but are not limited to laminin α2, dysferlin and calpain 3.

In one embodiment of the invention, the exon that is prevented from being present in the final, mature mRNA is an exon that includes a proteolytic cleavage recognition site, although this need not always be the case. Exon skipping may be used to eliminate sequences encoding any unwanted attribute of a protein. With respect to the removal of proteolysis recognition sites or sequences, one example of such a site is the four amino acid sequence “HPSS” located in repeat 1.6 of exon 42. Thus, the elimination of exon 42 is beneficial in stabilizing Δ45-47 and Δ45-48 dystrophin proteins, as demonstrated herein. However, those of skill in the art will recognize that other protease recognition sequences may be present on other exons, which might also be targeted, examples of which include but are not limited to dystrophin hinges: hinge 1 (exons 8 and 9), hinge 2 (exons 17 and 18), hinge 3 (exons 50 and 51) and hinge 4 (exons 61 to 64, but not in frame and consequently deletion of exons 61 to 67 or 59 to 64 is necessary), or by removal of exons 70 to 75 (proteolytic recognition site is located in exons 73 to 75 but not in frame and therefore deletion of exons 70 to 75 is necessary).

Those of skill in the art will also understand that the removal of exons should generally be carried out conservatively and that preferably, non-essential exons will be targeted. With respect to the dystrophin protein, exons that may be considered non-essential generally include any exon or combinations of exons which if deleted result in maintenance of the reading frame and a clinical phenotype of BMD (Tuffery-Giraud S et al, 2009). According to the present invention, one or more than one of these exons may be removed in order to promote stability of Δ45-47 and Δ45-48 dystrophin proteins or other partially deleted or inserted or mutation-bearing dystrophin proteins. If more than one exon is removed, the exons that are removed may be contiguous (located next to one another in primary sequence, e.g. exons 40 and 41 or exons 41 and 42) or they may not be contiguous (e.g. exons 40 and 42 may be removed, or exons 40, 41, 42 and 48 may be removed, etc.), as long as the resulting mRNA retains a correct open reading frame. Further, more than one exon refers to two or more, e.g. 3, 4, 5, 6, 7, 8, 9, 10 or more exons that may be beneficially removed. Examples from the existing databases show that large deletions such as of exons 2-7 or of exons 13-29 may be associated with mild BMD phenotypes (Tuffery-Giraud, S. et al, 2009). Those of skill in the art will recognize that the selection of exons for removal as described herein will usually be predicated on the expectation of a beneficial result such as stabilization of the protein, e.g. by removal of a proteolytic recognition site.

Generally, the removal of exon-encoded sequences from a dystrophin protein is carried out using anti-sense oligonucleotides (AONs). Oligonucleotides are designed to complement suitable sequences, usually RNA sequences within the pre-mRNA molecule which are required for correct splicing of the targeted exon(s), thereby blocking splicing reactions that would incorporate the targeted exon(s) into mature mRNA. An AON typically binds to the sequence which it complements and sterically hinders the splicing reaction. Sequences are selected so as to be specific, i.e. the AON's are complementary only to the sequences of the pre-mRNA and not to other nucleic acid sequences. The AON's used in the practice of the invention may be of any suitable type, e.g. oligodeoxyribonucleotides, oligoribonucleotides, morpholinos, tricyclo-DNA-antisense oligonucleotides, U7- or U1-mediated. AONs or conjugate products thereof such as peptide-conjugated or nanoparticle-complexed AONs. AONs employed in the practice of the invention are generally from about 10 to about 30 nucleotides in length, and may be for example, about 10 or fewer, or about 15, or about 20 or about 30 nucleotides or more in length. The binding affinity of the AON's for a targeted complementary sequence is generally in the range of from about 15 to about 25 nucleotides long depending on the chemical backbone used and on the target sequence. Typically, morpholino-AONs are about 25 nucleotides long, 2′PMO-AONs are about 20 nucleotides long, and tricyclo-AONs are about 15 nucleotides long.

For use in the instant invention, the AON's of the invention can be synthesized de novo using any of a number of procedures well known in the art. For example, the b-cyanoethyl phosphoramidite method (Beaucage, S. L., and Caruthers, M. H., Tet, Let. 22:1859, 1981); nucleoside H-phosphonate method (Garegg et al., Tet. Let, 27:4051-4054, 1986; Froehler et al., Nucl. Acid. Res, 14:5399-5407, 1986,; Garegg et al., Tet. Let. 27:4055-4058, 1986, Gaffney et al., Tet, Let. 29:2619-2622, 1988). These chemistries can be performed by a variety of automated nucleic acid synthesizers available in the market. These nucleic acids may be referred to as synthetic nucleic acids. Alternatively, AON's can be produced on a large scale in plasmids, (see Sambrook, T., et al., “Molecular Cloning: A Laboratory Manual”, Cold Spring Harbor laboratory Press, New York, 1989). AON's can be prepared from existing nucleic acid sequences using known techniques, such as those employing restriction enzymes, exonucleases or endonucleases. AON's prepared in this manner may be referred to as isolated nucleic acids.

For use in vivo, the AON's may be are stabilized, A “stabilized” AON refers to an AON that is relatively resistant to in vivo degradation (e.g. via an exo- or endo-nuclease) Stabilization can be a function of length or secondary structure. Alternatively, AON stabilization can be accomplished via phosphate backbone modifications. Preferred stabilized AON's of the instant invention have a modified backbone, e.g. have phosphorothioate linkages to provide maximal activity and protect the AON from degradation by intracellular exo- and endo-nucleases. Other possible stabilizing modifications include phosphodiester modifications, combinations of phosphodiester and phosphorothioate modifications, methylphosphonate, methylphosphorathioate, phosphorodithioate, p-ethoxy, and combinations thereof. Chemically stabilized, modified versions of the AON's also include “Morpholinos” (phosphorodiamidate morpholino oligomers, PMOs), 2′-O-Met oligomers, tricyclo (tc)-DNAs, U7 short nuclear (sn) RNAs, or tricyclo-DNA-oligoantisense molecules (U.S. Provisional Patent Application Ser. No. 61/212,384 For: Tricylco-DNA Antisense Oligonucleotides, Compositions and Methods for the Treatment of Disease, filed. Apr. 10, 2009, the complete contents of which is hereby incorporated by reference. Other forms of AONs that may be used to this effect are AON sequences coupled to small nuclear RNA molecules such as U1 or U7 in combination with a viral transfer method based on, but not limited to, lentivirus or adeno-associated virus (Denti, M A, et al, 2008; Goyenvalle, A, et al, 2004).

The invention provides methods for treating a patient or individual that has or is suffering from disease symptoms that can be alleviated by employing the technique of exon skipping, particularly if the symptoms are caused by a lack of stability or function in a protein of interest that is encoded by a pr-mRNA containing multiple exons and introns. The invention provides methods to stabilize the protein of interest in the patient, especially when the lack of stability is due to an exposed or accessible proteolytic cleavage site that can be removed or eliminated by exon skipping. In some embodiments, the individual who is treated displays symptoms of BMD and the protein(s) of interest is/are one or both of the Δ45-47 and Δ45-48 dystrophin proteins. In another embodiment, the individual who is treated is a DMD patient who as a result of another treatment such as exon skipping or stop codon readthrough expresses a semifunctional and protease-sensitive dystrophin molecule. In order to carry out the methods of the invention, generally at least one, and usually from about 2 to 5 or more AONs are administered to such a patient in order to cause exon skipping of one or more exons of interest (e.g. exon 42) during pre-mRNA splicing. To that end, the invention also provides pharmaceutically acceptable (i.e. physiologically compatible) compositions comprising one or more AONs specifically complementary to nucleic acid sequences that are necessary for correct splicing and inclusion of the one or more exons of interest in mRNA, so that upon administration of the composition, inclusion of the one or more exons of interest in mRNA is blocked or prevented.

In addition to AONs, pharmaceutical compositions of the present invention may also include a pharmaceutically or physiologically acceptable carrier such as saline, sodium phosphate, etc. The compositions will generally be in the form of a liquid, although this need not always be the case. Suitable carriers, excipients and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, celluose, water syrup, methyl cellulose, methyl and propylhydroxybenzoates, mineral oil, etc. The formulations can also include lubricating agents, wetting agents, emulsifying agents, preservatives, buffering agents, etc. In particular, the present invention involves the administration of AONs and is thus somewhat akin to gene therapy. Those of skill in the art will recognize that nucleic acids are often delivered in conjunction with lipids (e.g. cationic lipids or neutral lipids, or mixtures of these), frequently in the form of liposomes or other suitable micro- or nano-structured material (e.g. micelles, lipocomplexes, dendrimers, emulsions, cubic phases, etc.).

The compositions of the invention are generally administered by injection, e.g. intravenously, subcutaneosly or intramuscularly, although other types of administration are not precluded, e.g. inhalation, topical, etc. Injectable preparations, for example, sterile injectable aqueous or oleaginous suspensions may be formulated according to the known art using suitable dispensing or wetting agents and suspending agents. The sterile injectable preparation can also be a sterile injectable solution or suspension in a nontoxic parenterally acceptable diluent or solvent, for example, as a solution in 1,3-butanediol. While delivery may be either local (i.e. in situ, directly into tissue such as muscle tissue) or systemic, usually delivery will be local to affected muscle tissue, e.g. to skeletal muscle, smooth muscle, heart muscle, etc. Depending on the form of the AONs that are administered and the tissue or cell type that is targeted, techniques such as electroporation, sonoporation, a “gene gun” (delivering nucleic acid-coated gold particles), etc. may be employed.

One skilled in the art will recognize that the amount of an AON to be administered will be an amount that is sufficient to induce amelioration of unwanted disease symptoms. Such an amount may vary inter alia depending on such factors as the gender, age, weight, overall physical condition, of the patient, etc. and may be determined on a case by case basis. The amount may also vary according to the type of condition being treated, and the other components of a treatment protocol (e.g. administration of other medicaments such as steroids, etc.). Generally, a suitable dose is in the range of from about 1 mgkg to about 100 mg/kg, and more usually from about 2 mg/kg to about 10 mg/kg. If a viral-based delivery of AONs is chosen, suitable doses will depend on different factors such as the viral strain that is employed, the route of delivery (intramuscular, intravenous, intraarterial or other), but may typically range from 10e10 to 10e12 viral particles/kg. Those of skill in the art will recognize that such parameters are normally worked out during clinical trials. Further, those of skill in the art will recognize that, while disease symptoms may be completely alleviated by the treatments described herein, this need not be the case. Even a partial or intermittent relief of symptoms may be of great benefit to the recipient. In addition, treatment of the patient is usually not a single event. Rather, the AONs of the invention will likely be administered on multiple occasions, that may be, depending on the results obtained, several days apart, several weeks apart, or several months apart, or even several years apart. This is especially true where the treatment of DMD or BMD is concerned since the disease is not cured by this treatment, i.e. the gene that encodes the protein will still be defective and the encoded protein will still possess an unwanted, destabilizing feature such as an exposed proteolytic recognition site, unless the AONs of the invention are administered.

With reference to the treatment of DMD patients with semi-functional dystrophin proteins that result from other exon skipping technologies, the methods of the present invention can be implemented in any of several different ways. For example, the AONs of the present invention may be administered together with AONs designed to remove other exons (e.g. in a single mixture, or in separate mixtures but administered in close temporal proximity, such as one directly after the other-in any order-with only a few minutes or hours between administrations). Alternatively, a patient who is already under treatment using e.g. exon skipping or stop codon readthrough protocols may be treated by the methods of the invention. In other words, the AONs of the invention may be administered to a patient who is already or has been receiving another treatment, but is still in need of further amelioration of the functional capabilities of the dystrophin molecules produced as a result of the other treatment.

If the AONs of the present invention are to be administered with AONs designed to skip exons for purposes other than to eliminate a protease recognition site, one possible route of administration is to include sequences encoding from both types of AONs (those designed to eliminate one or more exons encoding one or more protease recognition sites and those designed to eliminate exons for another reason) in a single vector that is administered to a patient. Those of skill in the art will recognize that several vectors are available for use in delivering nucleic acid sequences so that the nucleic acid sequences may be transcribed in vivo within the recipient. Examples of such vectors include but are not limited to various vectors derived from attenuated viruses such as retroviral vectors, adenoviral vectors, adeno-associated viral vectors, HIV and influenza virus vectors, etc. Vectors based on attenuated bacteria might also be employed, e.g. mycobacterial based vectors. Those of skill in the art will recognize that if these types of methods are used, it may be preferable to avoid multiple administrations which could result in an adverse immune response to the vector.

The individuals or patients treated by the methods described herein are typically mammals, usually humans. However, this need not always be the case. Veterinary applications of this technology are also contemplated.

The foregoing Examples serve to further illustrate the invention but should not be interpreted so as to limit the invention in any way.

### EXAMPLES

### Example 1

Duchenne muscular dystrophy (DMD) and Becker muscular dystrophy (BMD) both result from mutations in the dystrophin gene. However, while DMD is one of the most severe myopathies, BMD is characterized by much milder symptoms due to the production by BMD individuals of some truncated dystrophins. The functionality of these truncated dystrophins depends either on the importance of the missing domains or on their stability. As a result, BMD exhibits a wide range of phenotypes, ranging from almost asymptomatic to fairly severe. Interestingly, about half of the BMD population displays Δ45-47 or Δ45-48 exon deletions. These patients present unambiguous symptoms frequently associated with severe cardiac dysfunctions, although the majority of them are still ambulatory at 50 years of age.

The 45-47 and Δ45-48 exon deletions lack several, spectrin-like repeats upstream of the hinge 3 region. Six cryptic proteolysis sites have been described in dystrophin (Hori et al., Biochem Biophys Res Comm, 1995, 209(3): 1062-7). These are sites in the hinge 1 and hinge 2 regions (cleavage is lessened by actin binding), the hinge 3 and hinge 4 regions, the C-terminal region (cleavage lessened by syntrophin interaction), and a highly protease specific site (HPSS) in repeat 16 of exon 42. Since cleavage at HPSS should give rise to a putative fragment of about 220 kDa, we hypothesized that this site might be more active when exons 45-47 or 45-48 are missing, since a 220 kDa dystrophin protein is detected in muscle homogenates of Δ45-47 and Δ45-48 dystrophin BMD individuals.

In order to investigate this hypothesis, and to develop an interventional gene correction therapy for these patients having Δ45-47 and Δ45-48 dystrophin mutations, we carried out the experiments described below.

### Materials and Methods

### Western Blot

Western Blot multiplex of muscle protein of Becker (BMD) patients was carried out as described previously (Deburgrave and al., 2007). Multiplex A includes NCL-DYS1, NCL-CALP-2C4, and NCL-a-sarc); multiplex B includes NCL-DYS2, NCL-CALP12A2, NCL-g-SARC, NCL-Hamlet (dysferlin) (Novocastra).

### Cell Culture

The cultures of myoblasts and primary fibroblasts come respectively from muscle and cutaneous biopsy of BMD patients from the cell bank of the Hospital Cochin. Reactives and medium culture are products GIBCO Invitrogen unless otherwise indicated.

Cells are dissociated by treatment of biopsies with collagenase type 1A (Sigma). Cells are cultured during proliferation in HamF10 medium containing 20% foetal calf serum (Sigma), 100 units/mL of penicillin, 100 μg/mL of streptomycin.

The medium for differentiation of myoblasts into myotubes is constituted of DMEM+Glutamax+4.5 g of glucose+pyruvate sodium (1 mM), 2% horse's serum, 10 μg/mL insulin (Sigma), 100 μg/mL human apo-transferrin (Sigma), 100 units/mL penicillin, and 100 μg/mL streptomycin. Cells must be cultured in differentiation medium for 15 days.

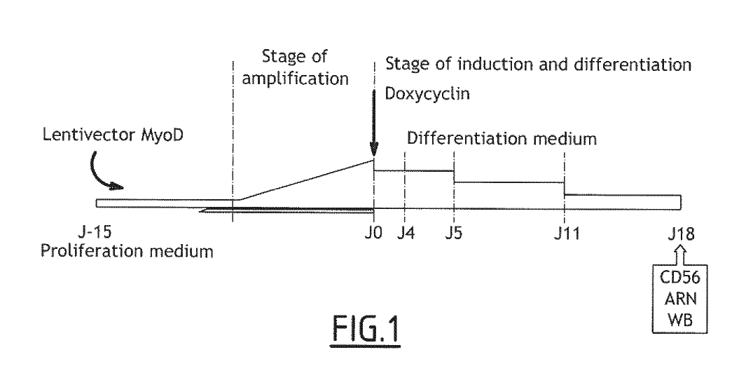

Skin fibroblasts cells were grown ex vivo and converted into myogenic progenitors by using a lentivector coding for an inducible MyoD gene with doxycyclin. About 600 to 1200 viral particles per cell were incubated with fibroblasts for at least 4 hours. This procedure is schematically represented in FIG. 1.

Transduced myoblasts and fibroblasts were cultivated in Petri dishes.

### Plasmid Construction

We have created a collection of plasmids coding for truncated dystrophins Δ45-47 and Δ45-48 with and without exon 42, and for a full-length dystrophin; eventually fused with GFP.

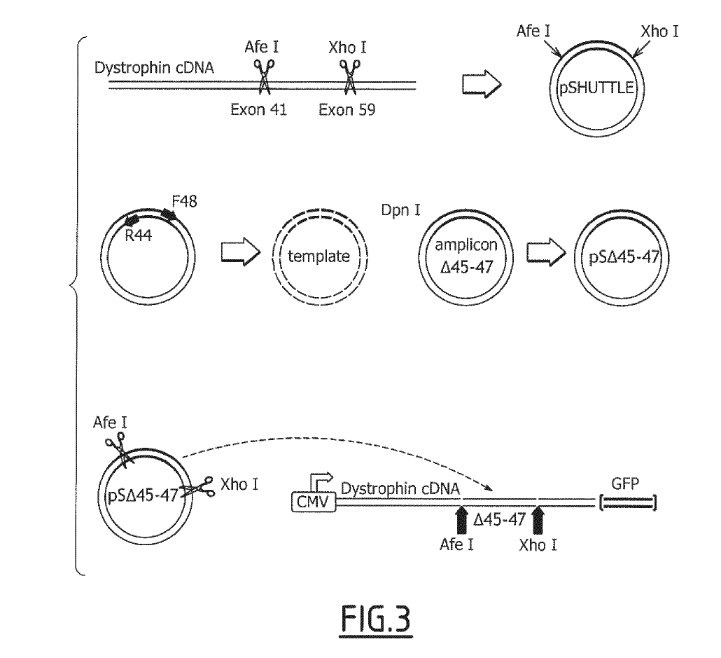

To build a Plasmid Shuttle: The full length human dystrophin cDNA was moved into the pCi plasmid (CMV promoter-MCS-Poly A). The resulting plasmid was digested by AfeI/XhoI in order to isolate and clone into pBSK a fragment of about 3 kb containing exons 42 to 58
[pShuttle]
To create deletions: Deletions were carried out by PCR mutagenesis in the pShuttle, as illustrated schematically in FIGS. 3A-C. We utilized five different primers, R45, F47, F48, R41, and F43, to create deletions. Forward primers contain a phosphate to permit ligations of pShuttle.

R45
(SEQ ID NO: 2)
CTTAAGATACCATTTGTATTTAGC
R41
(SEQ ID NO: 3)
AATTTGTGCAAAGTTGAGTCTTCG
F47
(SEQ ID NO: 4)
pGTTTCCAGAGCTTTACCTGAGAAAC
F48
(SEQ ID NO: 5)
pAAGGAAACTGAAATAGCAGTTCAAG
F43
(SEQ ID NO: 6)
pAATATAAAAGATAGTCTACAACAAAG

Final constructs Δ45-47, Δ45-48: AfeI/XhoI fragments from pSDi-j were brought back into the original pCi dystrophin expression vectors by ligation. Final constructs Δ42, Δ45-47; Δ42, Δ45-48: AfeI/XhoI fragments from pSDi-j were brought back, the final construct is obtained by homologous recombination with pCi Swa1 in bacteria BJ5183.

### Antisense in Exon 42

We designed 4 antisense sequences which target different exonic splicing enhancers (ESEs) in exon 42 of human dystrophin: first position −10 at +30 amino acid (SF2/ASF), second at position 80 at 120 aa (Srp40/55), third at position 100 at 142(Srp40/SC35), and the last at position 134 at 174 (SC35).

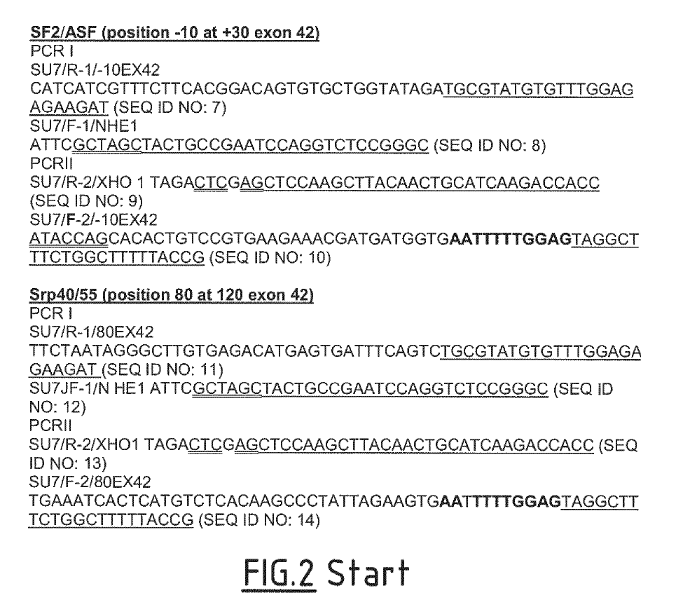

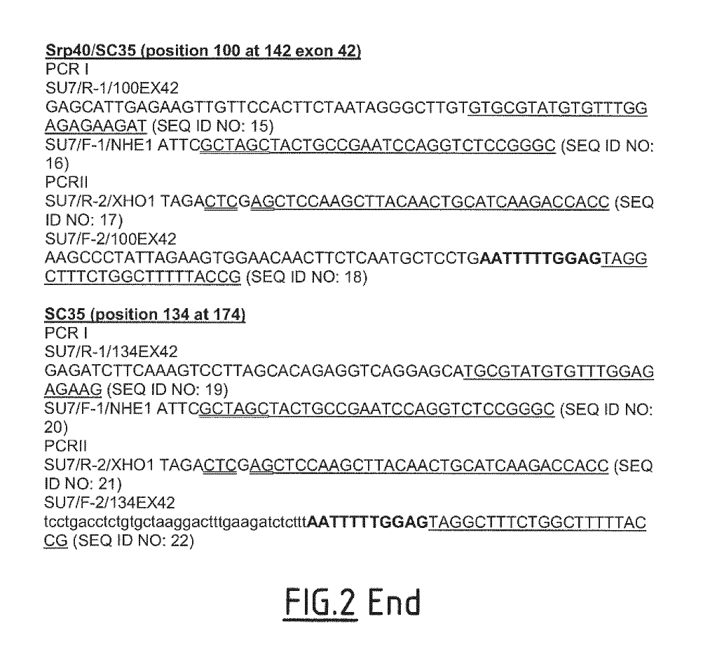

These antisense are introduced into the SU7smOPT gene by PCR as described previously (Govenvalle and al., 2004). The primers used are listed in the supplementary data in FIG. 2. These SU7-ESE antisense are introduced in the pRR1 vector with NheI and XbaI sites restriction. These antisense are also used in 2′O-methyl and tricyclo (tc)-DNA transfections.

2′O-methyl and tcDNA Transfection

We tested two different concentrations (10 μg and 50 μg) of four antisense oligonucloetides on myoconverted fibroblasts or myoblasts from Δ45-47 BMID) patients. We added DMEM only to final volume of 90 μL and incubated the mixture for 20 minutes with 12 μL of oligofectamin. 800 μL DMEM were then added and the cells were transferred to Petri dishes. Cells were then incubated for 4 h at 37° C. before adding 1 mL of DMEM +20% foetal calf's serum +1% penicillin-streptomycin. When iyotubes were observed, the cells were collected to carry out RT-PCR and western blotting or to conduct dystrophin immunoblotting.

### Zebrafish Experiments

pCi hDys Δ42, Δ45-47+GFP or Δ42, Δ45-Δ48+GFP were injected into zebrafish eggs. Embryos (24 h) were analyzed under confocal microscopy (4D) to test expression, localization, stability, turnover and functionality of these truncated dystrophins.

### Results

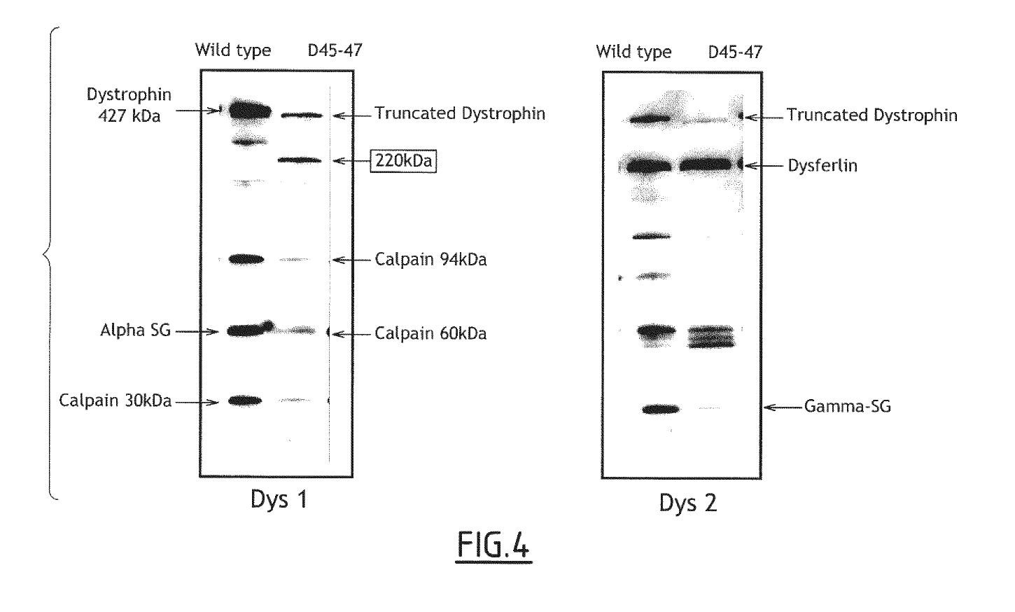

As can be seen in FIGS. 4A and B, the multiplex western blot of dystrophin from BMD patients Δ45-47, shows a degraded dystrophin at 220 kDa (FIG. 4A). A band of this molecular weight is also observed when dystrophin from BMD patients Δ45-48 is tested (FIGS. 4C and D).

A search of the amino acid sequence revealed the sequence HPSS in repeat 16 of exon 42. We hypothesized that proteolytic cleavage of Δ45-47 or Δ45-48 dystrophin at this site would give rise to a putative fragment of about 220 kDa, consistent with the results obtained with multiplex western blotting of dystrophin described in the previous paragraph.

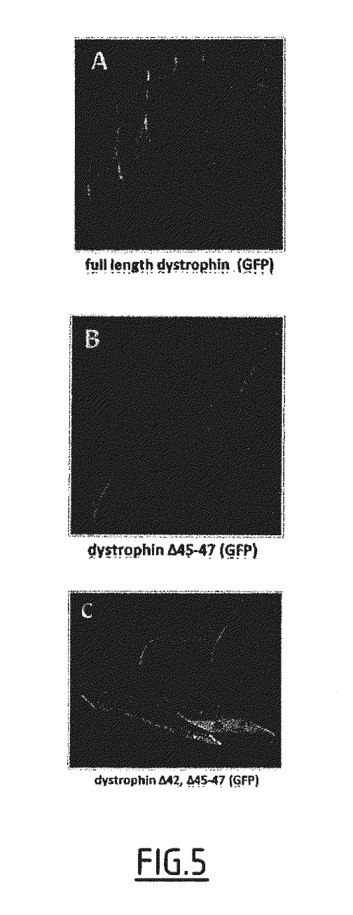

To test whether the stability of a Δ45-47 or a Δ45-48 dystrophin molecule would be improved if the amino acid sequences encoded by exon 42 (including the HPSS site) were removed, we injected constructs of truncated dystrophin fused with green fluorescent protein (GFP) into zebrafish eggs. The results showed that Δ45-47 or Δ45-48 dystrophin without exon 42, and thus without HPPS, seem to be more stable. As shown in FIG. 5, large quantities of Δ42, Δ45-47 dystrophin were localized at the tips of muscle fibers.

This experiment indicates that the Δ42, Δ45-47 and Δ42, Δ45-48 dystrophin proteins are more stable than their parent molecules (Δ45-47 and Δ45-48) as a result of removing exon 42. Therefore, removing the exon 42 from pre-mRNA of individuals with these deletion mutations using an exon skipping strategy is likely to be a good therapeutic approach for these patients.

### Example 2

Experiments similar to those described in Example 1 will also be carried out in dystrophin knock-out (KO) zebrafish and in the mdx mouse model. The results will show that the presence of the Δ42, Δ45-47 and Δ42, Δ45-Δ48 proteins can compensate for the loss of the dystrophin protein.

Fluorescence Recovery after Photobleaching (FRAP) experiments will also be carried, out to document protein turnover and relocalization of the truncated dystrophin proteins. The results will show normal or near normal protein turnover and localization of the Δ42, Δ45-47 and Δ42, Δ45-648 proteins

### Example 3

In vivo exon skipping of exon 42 in Δ45-47 and Δ45-Δ48 dystrophin deletion mutation: corrective gene therapy for BMD patients

AONs complementary to sequences necessary for correct splicing of exon 42 in the dystrophin gene are designed and prepared, e.g. phosphorodiamidate morpholino oligomers (PMOs), 2′-O-Met oligomers, tricyclo (tc)-DNAs, U7 short nuclear (sn) RNAs, etc.). The AONs bind to dystrophin pre-mRNA within (internal to) or near (eternal to) exon 42 of the dystrophin gene. When these AONs are administered to individuals suffering from BMD due to either a Δ45-47 deletion mutation or a Δ45-Δ48 deletion mutation in the dystrophin gene, pre-mRNA processing is perturbed and exon 42 is not incorporated into the mature mRNA The protein that is translated in the muscle cells of these individuals thus lacks sequences encoded by exon 42, and thus the protease recognition site HPSS is absent, and the resulting Δ42, Δ45-47 and Δ42, Δ45-Δ48 proteins are produced instead. These “corrected” proteins are significantly more stable than Δ45-47 and Δ45-Δ48 deletion mutants, and function normally in muscle cells of the recipients. This results in an attenuation of disease symptoms and improvement in muscle-related activities of the patients.

### Example 4

In vitro exon skipping will be performed on myoblast cell lines of DMD patients carrying deletions of exon 50 or exons 49-50. These cell lines will in a first step be treated by skipping exon 51 using AONs, thereby restoring the open reading frame and leading to the expression of a semifunctional Δ50-51 or Δ49-51 dystrophin protein which displays increased sensitivity to protease-mediated proteolysis. In a second step the expression of this semifunctional Δ50-51 or Δ49-51 dystrophin molecule will be stabilized by introducing an additional Δ42 deletion using AONs.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Barton-Davis E R, Cordier L, Shoturma D I, Leland S E, Sweeney H L. Aminoglycoside antibiotics restore dystrophin function to skeletal muscles of mdx mice. J Clin Invest. 1999 August; 104(4):375-8
Deburgrave, N., F. Daoud, S. Llense, JC. Barbot, D. Récan, C. Peccate, A. H. M. Burghes, C. Béroud, L. Garcia, JC. Kaplan, J. Chelly, F. Letureq, Protein- and mRNA-based phenotype-genotype correlations in DMD/BMD with point mutations and molecular basis for BMD with nonsense and frameshift mutations in the DMD gene, Human Mntation, 28(2) (2007), 183-195
Denti M A, Incitti T, Sthandier O, Nicoletti C, De Angelis F G. Rizzuto E, Auricchio A, Musaró A, Bozzoni I. Long-term benefit of adeno-associated virus/antisense-mediated exon skipping in dystrophic mice. Hum Gene Ther. 2008 June; 19(6):601-8.
Goyenvalle, A., A. Vulin, F. Fougerousse, F. Letureq, J. C. Kaplan, L. Garcia and O, Danos, Rescue of dystrophic muscle through u7 snRNA-mediated exon skipping, Science 306 (2004), no. 5702, 1796-1799.
Gregorevic P, Allen J M, Minami B, Blankinship M J, Haraguchi M, Meuse L, Finn E, Adams M E, Froehner S C. Murry C E, Chamberlain J S, rAAV6-microdystrophin preserves muscle function and extends lifespan, in severely dystrophic mice. Nat Med. 2006 July; 12(7):787-9. Epub 2006 Jul. 2.
Kinali M, Arechavala-Gomeza V, Feng L, Cirak S, Hunt D, Adkin C, Guglieri M, Ashton E, Abbs S, Nihoyannopoulos P, Garralda M E, Rutherfbrd M, McCulley C, Popplewell L, Graham I R, Dickson O, Wood M J, Wells D J, Wilton S D, Kole R, Straub V, Bushby K, Sewry C, Morgan J E, Muntoni F. Local restoration of dystrophin expression with the morpholino oligomer AVI-4658 in Duchenne muscular dystrophy: a single-blind, placebo-controlled, dose-escalation, proof-of-concept study, Lancet Neurol 2009 Aug. 25. [Epub ahead of print]
Li S, Kimura E, Fall B M, Reyes M, Angello J C, Welikson R, Hauschka S D, Chamberlain J S. Stabe transduction of myogenic cells with lentiviral vectors expressing a minidystrophin. Gene Ther. 2005 July; 12(14): 1099-108
Tuffery-Giraud S, Beroud C, Letureq F, Yaou R B, Hamroun D, Michel-Calemard L, Moizard M P, Bernard R, Cossée M., Boisseau P, Blayau M, Creveaux I, Guiochon-Mantel A, de Martinville B, Philippe C, Monnier N, Bieth E, Van Kien P K, Desmet F O, Humbertclaude V. Kaplan J C, Chelly J, Claustres M. Genotype-phenotype analysis in 2,405 patients with a dystrophinopathy using the UMD-DMD database: a model of nationwide knowledgebase. Hum. Mutat, 2009 June; 30(6):934-45.
van Deutekom J C, Janson A A, Ginjaar I B, Frankhuizen W S, Aartsma-Rus A, Bremmer-Bout M, den Dunnen J T, Koop K, van der Kooi A J, Goemans N M, de Kimpe S J, Ekhart P F, Venneker E H, Platenburg G J, Verschuuren J, van Ommen. GJ. Local dystrophin restoration with antisense oligonucleotide PRO51. N Engl J Med. 2007 Dec. 27; 357(26): 2677-86.
Welch E M, Barton E R, Zhuo J, Tomizawa Y, Friesen W J, Trifillis P, Paushkin S, Patel M., Trotta C R, Hwang S, Wilde R G, Karp G, Takasugi J, Chen G, Jones S, Ren H, Moon Y C, Corson D, Turpoff A A, Campbell J A, Conn M M, Khan A, Almstead N G, Hedrick J, Mollin A, Risher N, Weetall. M, Yeh S, Branstrom A A, Colacino J M, Babiak J, Ju W D, Hirawat S, Northcutt V J, Miller L L, Spatrick P, He F, Kawana M, Feng H, Jacobson A, Peltz S W, Sweeney H L. PTC 124 targets genetic disorders caused by nonsense mutations, Nature, 2007 May 3; 447(7140):87-91. Epub 2007 Apr. 22.

1. A method for treating a muscular dystrophy caused by instability of a semi-functional dystrophin in a patient in need thereof, comprising the step of
administering to said patient antisense oligonucleotides complementary to nucleic acid sequences that are necessary for correct splicing of a region comprising or consisting of exon 42 of said semi-functional dystrophin.

2. The method of claim 1, wherein said antisense oligonucleotides are complementary to nucleic acid sequences within pre-mRNA encoding said exon 42.

3. The method of claim 1, wherein said antisense oligonucleotides are complementary to nucleic acid sequences adjacent to pre-mRNA encoding said exon 42, said nucleic acid sequences adjacent to pre-mRNA encoding said exon 42 being required for correct splicing of said exon 42.

4. The method of claim 1, wherein said administered antisense oligonucleotides prevent cleavage of said semi-functional dystrophin at a protease recognition sequence HPSS.

5. The method of claim 1, wherein said disease is Becker Muscular Dystrophy.

6. The method of claim 5, wherein said semi-functional dystrophin is selected from Δ45-47 dystrophin and Δ45-48 dystrophin.

7. The method of claim 1, wherein said disease is Duchenne Muscular Dystrophy with treatment-induced semifunctional dystrophin expression.

8. The method of claim 7, wherein said semifunctional dystrophin expression is induced by exon skipping treatment.

9. The method of claim 7, wherein said semifunctional dystrophin expression is induced by stop codon readthrough treatment.

10. The method of claim 1, wherein said antisense oligonucleotides are administered to muscle tissue of said patient.

11. The method of claim 10, wherein said muscle tissue is selected from skeletal muscle tissue, smooth muscle tissue and cardiac muscle tissue.

12. A method of stabilizing a semi-functional dystrophin protein comprising the step of
preventing cleavage of said semi-functional dystrophin protein at a protease recognition site HPSS.

13. The method of claim 12, wherein said step of preventing is carried out by blocking splicing of a region consisting of or comprising exon 42 of said semi-functional dystrophin.

14. The method of claim 12, wherein said semi-functional dystrophin protein is selected from the group consisting of Δ45-47 dystrophin protein, Δ45-48 dystrophin protein, Δ49-51 dystrophin protein and Δ50-51 dystrophin protein.

15. An antisense oligonucleotide complementary to
nucleic acid sequences of exon 42 of dystrophin, or
nucleic acid sequences adjacent to exon 42 which are required for correct splicing of exon 42 of dystrophin.

16. The antisense oligonucleotide of claim 15, wherein said antisense oligonucleotide is an oligomer selected from the group consisting of a phosphorodiamidate morpholino oligomer (PMO), a 2′-O-Met oligomer, a tricyclo (tc)-DNA oligomer, and a U1 or U7 short nuclear (sn) RNA oligomer.

17. A dystrophin protein selected from the group consisting of Δ42, Δ45-47 dystrophin protein; Δ42, Δ45-48 dystrophin protein; Δ42, Δ49-51 dystrophin protein; and Δ42, Δ50-51 dystrophin protein.

18. A pharmaceutical composition for the treatment of a Becker or Duchenne Muscular Dystrophy, comprising
the antisense oligonucleotide of claim 15, and
a pharmaceutically or physiologically acceptable carrier.

19-20. (canceled)

21. A method of manufacturing a medicament to treat a muscular dystrophy caused by instability of a semi-functional dystrophin, comprising the steps of
designing an antisense oligonucleotide complementary to
nucleic acid sequences of exon 42 of dystrophin, or
nucleic acid sequences adjacent to exon 42 which are required for correct splicing of exon 42 of dystrophin;
synthesizing said antisense oligonucleotide; and
combining said antisense oligonucleotide and a pharmaceutically or physiologically acceptable carrier to form said medicament.

22. The method of claim 21, wherein said muscular dystrophy is a Becker or Duchenne Muscular Dystrophy.


SEQUENCE LISTING (22 sequences)

SEQ ID NO: 1 - protein, 4 aa, Artificial Sequence
  protein recognition sequence
HPSS

SEQ ID NO: 2 - DNA, 24 bp, Artificial Sequence
  primer
cttaagataccatttgtatttagc

SEQ ID NO: 3 - DNA, 24 bp, Artificial Sequence
  primer
aatttgtgcaaagttgagtcttcg

SEQ ID NO: 4 - DNA, 25 bp, Artificial Sequence
  primer
gtttccagagctttacctgagaaac

SEQ ID NO: 5 - DNA, 25 bp, Artificial Sequence
  primer
aaggaaactgaaatagcagttcaag

SEQ ID NO: 6 - DNA, 26 bp, Artificial Sequence
  primer
aatataaaagatagtctacaacaaag

SEQ ID NO: 7 - DNA, 61 bp, Artificial Sequence
  oligonucleotide
catcatcgtttcttcacggacagtgtgctggtatagatgcgtatgtgtttggagagaagat

SEQ ID NO: 8 - DNA, 35 bp, Artificial Sequence
  oligonucleotide
attcgctagctactgccgaatccaggtctccgggc

SEQ ID NO: 9 - DNA, 40 bp, Artificial Sequence
  oligonucleotide
tagactcgagctccaagcttacaactgcatcaagaccacc

SEQ ID NO: 10 - DNA, 70 bp, Artificial Sequence
  oligonucleotide
ataccagcacactgtccgtgaagaaacgatgatggtgaatttttggagtaggctttctggctttttaccg

SEQ ID NO: 11 - DNA, 61 bp, Artificial Sequence
  oligonucleotide
ttctaatagggcttgtgagacatgagtgatttcagtctgcgtatgtgtttggagagaagat

SEQ ID NO: 12 - DNA, 35 bp, Artificial Sequence; identical to SEQ ID NO: 8
  oligonucleotide

SEQ ID NO: 13 - DNA, 40 bp, Artificial Sequence; identical to SEQ ID NO: 9
  oligonucleotide

SEQ ID NO: 14 - DNA, 70 bp, Artificial Sequence
  oligonucleotide
tgaaatcactcatgtctcacaagccctattagaagtgaatttttggagtaggctttctggctttttaccg

SEQ ID NO: 15 - DNA, 63 bp, Artificial Sequence
  oligonucleotide
gagcattgagaagttgttccacttctaatagggcttgtgtgcgtatgtgtttggagagaagat

SEQ ID NO: 16 - DNA, 35 bp, Artificial Sequence; identical to SEQ ID NO: 8
  oligonucleotide

SEQ ID NO: 17 - DNA, 40 bp, Artificial Sequence; identical to SEQ ID NO: 9
  oligonucleotide

SEQ ID NO: 18 - DNA, 72 bp, Artificial Sequence
  oligonucleotide
aagccctattagaagtggaacaacttctcaatgctcctgaatttttggagtaggctttctggctttttaccg

SEQ ID NO: 19 - DNA, 59 bp, Artificial Sequence
  oligonucleotide
gagatcttcaaagtccttagcacagaggtcaggagcatgcgtatgtgtttggagagaag

SEQ ID NO: 20 - DNA, 35 bp, Artificial Sequence; identical to SEQ ID NO: 8
  oligonucleotide

SEQ ID NO: 21 - DNA, 40 bp, Artificial Sequence; identical to SEQ ID NO: 9
  oligonucleotide

SEQ ID NO: 22 - DNA, 70 bp, Artificial Sequence
  oligonucleotide
tcctgacctctgtgctaaggactttgaagatctctttaatttttggagtaggctttctggctttttaccg